(19) United States
(12) Patent Application Publication
(10) Pub. No.: US 2018/0360419 A1
(43) Pub. Date: Dec. 20, 2018

(54) FINGER-MOUNTED ULTRASOUND PROBE ARRAY WITH VARIED CENTER FREQUENCIES

(71) Applicant: Whale Imaging, Inc., Waltham, MA (US)

(72) Inventors: Xun Zhu, Waltham, MA (US); Changguo Ji, Lexington, MA (US); William Wong, Milton, MA (US); Xingbai He, Belmont, MA (US)

(21) Appl. No.: 15/624,285

(22) Filed: Jun. 15, 2017

(51) Int. Cl. A61B 8/00; G01S 15/89; G01S 7/52

(52) U.S. Cl. CPC A61B 8/4455; A61B 8/4477; G01S 7/52079; G01S 15/8936

(57) ABSTRACT

A single hand operation ultrasound probe array is provided. The single ultrasound probe array places individual transducers, each having different transducer characteristics, on sheaths that fit over fingers of a user. By the user positioning their fingers differently, different transducers are selected for use, enabling the user to switch between multiple different center frequencies, or other different transducer configurations, based on the positioning of the fingers of the user relative to the patient. The ultrasound probe array is configured with wired or wireless communication to an ultrasonic machine to receive the probe signals and interpret.

### FIELD OF THE INVENTION

The present invention relates to an ultrasound probe suitable for single hand operation. In particular, the present invention relates to a multiple frequency single hand ultrasound probe configured to adjust the selection of transducer, and thereby transducer characteristics such as frequency, based on the positioning of the fingers of the user relative to the patient.

### BACKGROUND

Generally, ultrasound imaging uses high frequency sound waves to provide direct visualization of various soft tissues of the body. As a result of the improved resolution of ultrasound images in recent years, nerves and nerve tissue can be well visualized. Additionally, bone edges can be identified with ultrasound such that joint spaces are quite obvious. Another benefit of ultrasound imaging is that the process does not expose the patient and healthcare provider to radiation risks associated with other imaging techniques (e.g., X-ray). Portable ultrasound machines have been developed to be smaller and lighter than console style ultrasound machines. Typically, mobile ultrasound systems can be carried by hand and in some cases, operate for a time on battery power alone. Portable ultrasound machines are typically used in situations where space is limited, mobility is important, and/or the scanning must be done in the field. Currently, portable ultrasound machines are used in Cardiac, Vascular, Radiology, Endocrinology, Pediatric, OB/GYN applications, and almost all kinds of surgeries.

As an imaging modality, ultrasound systems are able to generate a real-time image and real-time image loops, or movies. Such real-time imaging is beneficial to medical professionals making diagnoses. Using real-time ultrasound imaging, a procedure may be documented and the actual motion that a patient goes through to demonstrate area of pain may be documented. For example, a shoulder impingement can be visualized as it occurs on an ultrasound loop. Another benefit of the improved performance of ultrasound-guided surgery is a decrease in health care costs per patient, while improving patient care. Several studies have been conducted to compare the efficacy of blindly guided techniques (landmark technique) versus ultrasound-guided surgeries. Those studies provide proof that ultrasound guided surgeries yield better patient results, and thus, incorporating diagnostic ultrasound imaging is a tool that improves physician confidence. In particular, when surgeons operate to remove a tumor, determining exactly where to cut can be tricky. Ideally, the entire tumor should be removed while leaving a continuous layer of healthy tissue, but locating the tumors without assisted image guiding (e.g., ultrasound) during surgery is difficult. Ultrasound-guided surgery is becoming increasingly important in improving cancer, plastic, and surgical treatments. At present, surgeons can utilize intra-operative ultrasound imaging to extract tumors with more precision and ease, while reducing trauma for the patient. The progressing of ultrasound technology and improved performance of ultrasound-guided surgery is leading a decrease in health care costs per patient, and at the same time, improving the efficiency and quality of patient care.

Mechanical construction of a typical ultrasonic instrumentation system includes parameters such as the radiation surface area, mechanical damping, housing, connector type and other variables of physical construction. The transducer is a vital part of an ultrasonic instrumentation system. The transducer incorporates a piezoelectric element, which converts electrical signals into mechanical vibrations (transmit mode) and mechanical vibrations into electrical signals (receive mode). Many factors influence the behavior, and characteristics, of a transducer, including material, mechanical and electrical construction, and the external mechanical and electrical load conditions. Some transducers are specially fabricated to be more efficient transmitters and others to be more efficient receivers. A transducer that performs well in one application will not always produce the desired results in a different application. For example, sensitivity to small defects is proportional to the product of the efficiency of the transducer as a transmitter and a receiver.

It is also important to understand the concept of bandwidth, or range of frequencies, associated with a transducer. The frequency noted on a transducer is the central or center frequency and depends primarily on the backing material. For example, highly damped transducers respond to frequencies above and below the central frequency. The broad frequency range provides a transducer with high resolving power. Less damped transducers exhibit a narrower frequency range and poorer resolving power, but greater penetration. The central frequency also defines the capabilities of a transducer. Lower frequencies provide greater energy and penetration in a material, while higher frequency crystals provide reduced penetration but greater sensitivity to small discontinuities. High frequency transducers, when used with the proper instrumentation, can improve flaw resolution and thickness measurement capabilities dramatically. Additionally, a higher frequency generates an ultrasound beam with energy that is more confined, and attenuates faster. For imaging, it means better resolution and reduced imaging depth. The selection of the center frequency of an ultrasound transducer is determined largely based on different probing purposes. Due to the different frequency applications, ultrasound guided biopsy and surgery, requires medical personnel to switch between probes with different center frequency in real-time, such as during the surgery, and/or adjust the center frequencies of a probe. Switching between probes has a number of shortcomings, including but not limited to an increased risk of cross-contamination and increased duration of time for a procedure.

### SUMMARY

There is a need for improvements to the operability, configurability, and utilization of ultrasonic devices/systems during ultrasound assisted procedures (e.g., guided biopsy and surgery). The present invention is directed toward further solutions to address this need, in addition to having other desirable characteristics. Specifically, a finger-mounted ultrasound probe array and method of use is provided for eliminating the need for complete exchanging between different probe devices and/or changing a center of frequency of a single probe. The elimination of such exchanges can greatly shorten the surgery time and decrease the risk of cross contamination caused by complete exchange of different probe devices. The finger-mounted ultrasound probe array can be utilized in the same manner as conventional ultrasound devices, for example, to assist in performing a biopsy, tumor extracting, spinal and all kinds of surgeries. The finger-mounted ultrasound probe array provides medical professionals with a convenient and comfortable method for change between different center frequencies of the ultrasonic system while probing without exchanging probe devices or stopping to switch a frequency setting on probe devices.

In accordance with example embodiments of the present invention, a finger-mounted ultrasound probe array is provided. The probe array includes a plurality of finger sheaths, each having a first end and a second end and each sized and dimensioned to fit over a finger of a user. The probe array also includes a plurality of transducers, each transducer coupled with each of the plurality of finger sheaths. Each transducer of the plurality of transducers produces a different center frequency from the other of the plurality of transducers.

In accordance with aspects of the present invention, the plurality of transducers are located proximate to a mid-region of each of the plurality of finger sheaths and are configured to fire and receive ultrasonic waves in a side firing orientation.

In accordance with aspects of the present invention, the plurality of transducers are located proximate to the second end of each of the plurality of finger sheaths and are configured to fire and receive ultrasonic waves in an end firing orientation. The plurality of transducers can be linear probes. The plurality of transducers can be convex probes. The plurality of transducers can be 2D matrix probes. The plurality of transducers can be piezoelectric transducers or capacitive transducers.

In accordance with aspects of the present invention, the plurality of finger sheaths are sized, dimensioned, and configured to slide over fingers of a user wearing a surgical glove. The plurality of finger sheaths can themselves be portions of a surgical glove. Each of the plurality of finger sheaths can be distinct and separate from each other of the plurality of finger sheaths. Each of the plurality of finger sheaths can be fixed together.

In accordance with aspects of the present invention, the probe array further includes communication hardware in communication with the probe array and configured for communication with an ultrasound machine using a wired or wireless form of communication. The probe array can be adapted to implement ultrasound procedures during a biopsy procedure, or a tumor extracting procedure.

In accordance with aspects of the present invention, each of the plurality of finger sheaths is removable and replaceable in real-time during use of the probe array to enable swapping in and out of transducers having different transducer characteristics. The plurality of transducers can include high frequency transducers. The plurality of transducers can include mid-range frequency transducers. The plurality of transducers can include low frequency transducers.

### BRIEF DESCRIPTION OF THE FIGURES

These and other characteristics of the present invention will be more fully understood by reference to the following detailed description in conjunction with the attached drawings, in which:

FIGS. 1A and 1B are diagrammatic illustrations of a plurality of finger sheaths having transducers, forming a finger-mounted ultrasound probe array, in accordance with the present invention;

FIG. 2 is an example depiction of an ultrasonic system utilizing the finger-mounted ultrasound probe array, in accordance with the present invention;

FIGS. 3A and 3B are diagrammatic illustrations of finger-mounted ultrasound probe arrays mounted on a user's hand, in accordance with the present invention;

FIGS. 4A and 4B are diagrammatic illustrations of the finger-mounted ultrasound probe array incorporated into a glove, in accordance with the present invention; and

FIG. 5 is a flowchart depicting an example method of operation of the ultrasonic device, in accordance with the present invention.

### DETAILED DESCRIPTION

An illustrative embodiment of the present invention relates to a finger-mounted ultrasound probe array for diagnostic applications. In particular, the present invention provides an ultrasound probe array configured with a plurality of transducers incorporated into finger sheaths. An operating user inserts their fingers into the finger sheaths and places one of the transducers on a patient during imaging. Based on the finger selected for placement on the patient, the imaging corresponds to the frequency associated with the transducer located on that finger. When the user wants to change the frequency during the imaging, the user merely has to switch which finger is placed on the patient, thereby causing contact between the transducer on that particular finger having specific transducer characteristics different from other transducers in the probe array. Depending on the number of finger sheaths and corresponding transducers, the user can have access to multiple different frequencies or other transducer characteristics and can rapidly change between their frequencies as fast as they can change their finger placement.

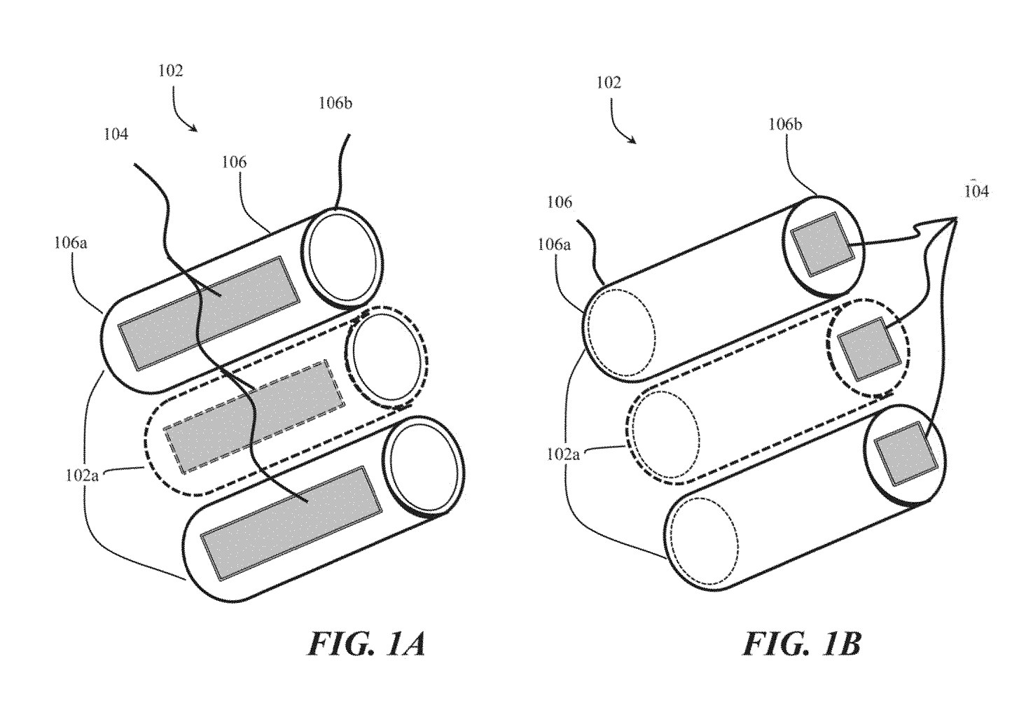

FIGS. 1A through 5, wherein like parts are designated by like reference numerals throughout, illustrate an example embodiment or embodiments of a finger-mounted ultrasound probe array having improved functionality, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

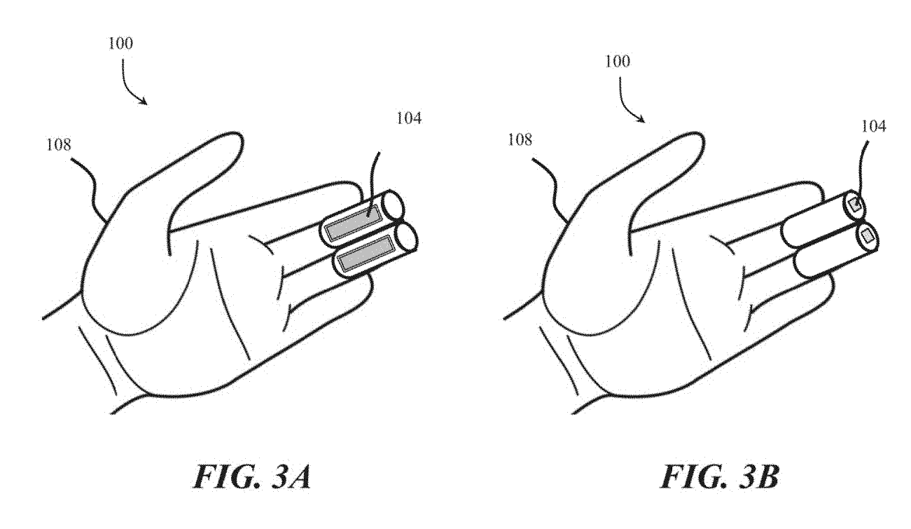

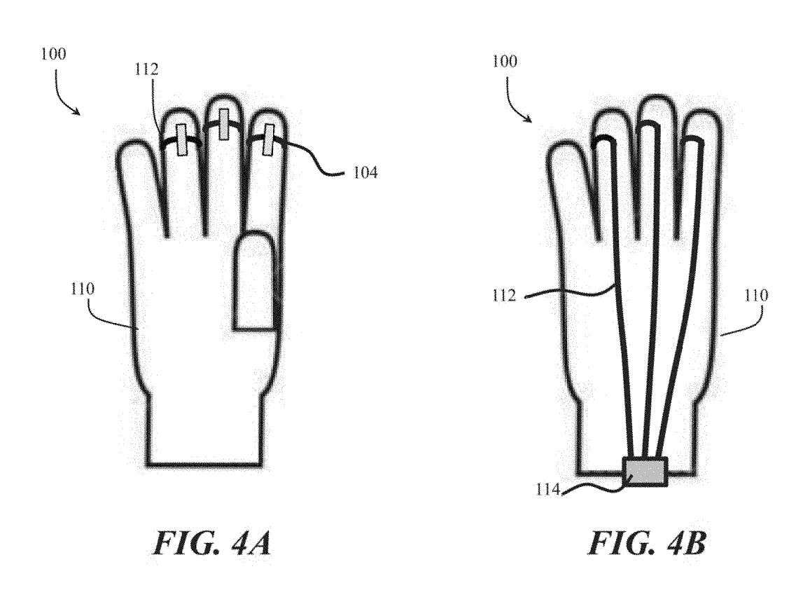

FIGS. 1A and 1B depict finger-mounted ultrasound probe arrays 102 configured to provide finger-mounted ultrasound probing capabilities. In particular, FIGS. 1A and 1B depict a plurality of transducers 104 combined with a plurality of finger sheaths 106 to form a plurality of probes 102a that form the finger-mounted ultrasound probe array 102. As would be appreciated by one skilled in the art, the probe array 102 can include any number of probes 102a. For example, the probe array 102 can include three probes 102a (as depicted in FIGS. 1A and 1B), two probes 102a (as depicted in FIGS. 3A and 3B), or any other number of probes that will fit on one or both hands of the user. The probe array 102 is configured for utilization as a finger-mounted ultrasound probing device 100 itself, as depicted in FIGS. 3A and 3B, and/or as a component of the finger-mounted ultrasound probing device 100, as depicted in FIGS. 4A and 4B.

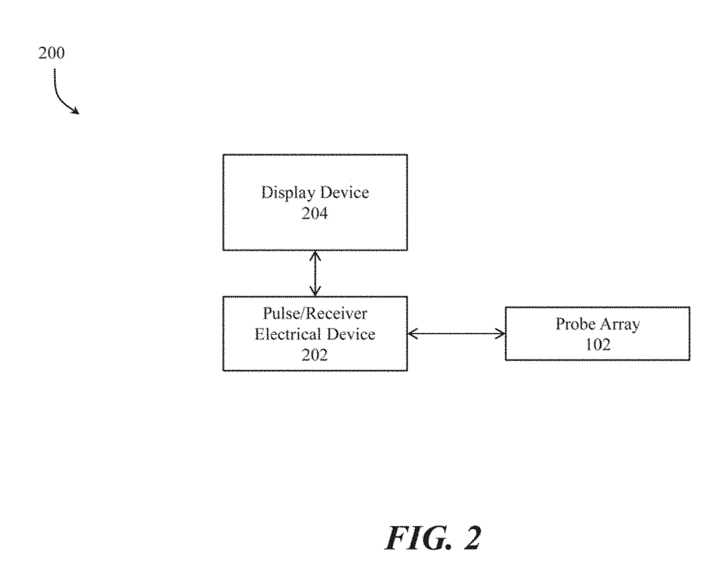

In accordance with an example embodiment of the present invention, the probe array 102 is configured to operate as part of or in communication with an ultrasonic system 200. In particular, the probe array 102 is configured to be an ultrasonic probe device for use with ultrasonic systems 200. When the probe array 102 is included within an ultrasonic system 200, as depicted in the example ultrasonic system 200 of FIG. 2, the probe array 102 is configured to be coupled to a pulse/receiver electrical device 202 and display device(s) 204 in a manner to enable ultrasound procedures. The pulse/receiver electrical device 202 is configured to generate pulses of voltage to be fed to the plurality of transducers 104 within the probe array 102. In response to the pulses of voltage, the plurality of transducers 104 generate center frequency ultrasonic energy waves. Depending on the object that the probe array 102 is placed on, the ultrasonic energy waves are reflected back to the plurality of transducers 104 and subsequently converted to voltage to be transmitted to the display device for display (via the pulse/receiver electrical device 202). As would be appreciated by one skilled in the art, the probe array 102 can configured for communication with the various components of an ultrasound machine (e.g., the pulse/receiver electrical device 202 and display device(s) 204) using a wired or wireless form of communication.

Continuing with FIGS. 1A and 1B, each of the plurality of probes 102a includes a finger sheath 106 with a transducer of the plurality of transducers 104 fixedly attached thereto. In accordance with an example embodiment of the present invention, each of the finger sheaths 106 is designed with a hollow cavity sized and dimensioned to fit over one or more fingers of a user. In particular, each of the finger sheaths 106 has a first end 106a and a second end 106b with the first end 106a including an opening sized and dimensioned to receive a finger of a user, and the second end 106b located most proximal a distal end of a user's finger (e.g., proximal the user's fingertips), as depicted in FIGS. 3A and 3B. In accordance with an example embodiment of the present invention, each of the finger sheaths 106 is distinct and separate from each other such that the fingers of the user can move independently during use. In another example each of finger sheaths 106 is fixed together such that the fingers must move together as a single unit. In operation, regardless of configuration, each of the finger sheaths 106 and/or a combination of finger sheaths 106 is configured for real-time removal and replacement from the probe array 102 during a surgical procedure. For example, a user can remove one finger sheath 106 including a transducer of the plurality of transducers 104 with one frequency (e.g., low frequency) and replace it with another finger sheath 106 including a transducer of the plurality of transducers 104 with another frequency (e.g., a high frequency).

In accordance with an example embodiment of the present invention, the plurality of transducers 104 are located proximal to a mid-region (between the first end 106a and the second end 106b) of each of the finger sheaths 106 and is configured to fire and receive ultrasonic waves in a side firing orientation, as depicted in FIG. 1A and 3A. In particular, the transducers 104 are located at a mid-region position of the finger sheaths 106 in an orientation such that the plurality of transducers 104 face the same direction as a palm of the user wearing the finger-mounted ultrasound probing device 100, as depicted in FIG. 3A. In accordance with an example embodiment of the present invention, the plurality of transducers 104 are located proximate to the second end 106b of each of the plurality of finger sheaths 106 and are configured to fire and receive ultrasonic waves in an end firing orientation, as depicted in FIG. 1B and 3B. In particular, the plurality of transducers 104 are located at the second end 106b of the finger sheaths 106 in an orientation such that the plurality of transducers 104 face outward from the fingertips of the user wearing the finger-mounted ultrasound probing device 100, as depicted in FIG. 3B. Additionally, the plurality of transducers 104 can be configured in a combination of shapes and sizes. For example, the plurality of transducers 104 depicted in FIG. 1A may be of a larger size and dimension from the plurality of transducers 104 depicted in FIG. 1B. In another example, the plurality of transducers 104 can also be flat or contoured to fit a user's fingers. As would be appreciated by one skilled in the art, a probe array can include a combination of transducers 104 from FIGS. 1A and 1B. For example, the probe array 102 can include transducers 104 with different firing directions on different fingers such that a user chooses the transducers 104 with a firing direction that is a specific need for a particular task.

In accordance with an example embodiment of the present invention, each of the plurality of transducers 104 within the probe array 102 is configured to produce different center frequencies (e.g., utilizing transducers with different backing material, different longitudinal dimension of crystal elements, etc.). For example, of the plurality of transducers 104 depicted in FIGS. 1A and 1B, one transducer can be a high frequency transducer (e.g., 8-20 MHz), one transducer can be a mid-range frequency transducer (e.g., 4-8 MHz), and one transducer can be a low frequency transducer (e.g., 2-4 MHz). As would be appreciated by one skilled in the art, any combination of high frequency transducers, mid-range frequency transducers, and low frequency transducers can be utilized within the probe array 102 without departing from the scope of the present invention. Furthermore, the range of each transducer of the plurality of transducers 104 can be specifically tuned to a given frequency or frequency range. For example, the probe array 102 can include three high frequency transducers of the plurality of transducers 104 with one tuned at 4-5 MHz, one tuned at 5-6 MHz, and one tuned at 6-8 MHz. Accordingly, the frequency of the plurality of transducers 104 can be customizable to the application in which the probe array 102 will be utilized. As would be appreciated by one skilled in the art, during operation, the high frequency transducers are utilized for fine resolution applications but are weak when it comes to penetration and provide a shallow scanning depth. Whereas low frequency transducers are utilized for rough resolution applications but are strong when it comes to penetration and provide a deep scanning depth.

Additionally, the plurality of transducers 104 utilized within the probe array 102 can include any type of transducers suitable for performing ultrasonic operations. In particular, the plurality of transducers 104 can include any type of sensor capable of generating acoustic signals and detecting returned signals. For example, the plurality of transducers 104 can include any combination of piezoelectric transducers, capacitive transducers, or any transducers known in the art.

The combination of the plurality of transducers 104 and finger sheaths 106 form the multiple probes 102a that create the probe array 102. The probe array 102 created by the plurality of probes 102a can also be configured to perform as different probe types. For example, the probe array 102 can be implemented as a linear probe, a convex probe, a phased probe, a single probe, a 2D matrix probe, etc. As would be appreciated by one skilled in the art, the finger-mounted ultrasound probing device 100 of the present invention can be implemented for utilization with three-dimensional imaging applications.

FIGS. 3A, 3B, 4A, and 4B depict example implementations of the probe array 102 to form the finger-mounted ultrasound probing device 100. FIGS. 3A and 3B depict the probe array 102, and finger sheaths 106, worn as the finger-mounted ultrasound probing device 100 itself. In the example embodiment provided by FIGS. 3A and 3B, each of the finger sheaths 106 (with the transducers attached thereto) in the probe array 102 slides over a different finger on a hand 108 of a user. FIG. 3A depicts a probe array 102 including side firing transducers, as discussed with respect to FIG. 1A. FIG. 3B depicts a probe array 102 including end firing transducers, as discussed with respect to FIG. 1B. The probe arrays 102 provided in FIGS. 3A and 3B can be worn on a bare hand of the user or over a glove (e.g., latex or other surgical grade gloves) worn by the user. In accordance with an example embodiment of the present invention, the probe array 102 is wired to an ultrasound system using any communication medium known in the art. For example, the probe array 102 of the present invention can be wired to an ultrasound machine by a hardware switch or wireless communication platform.

FIGS. 4A and 4B depict different diagrammatic illustrations of the probe array 102 implemented as a component of the finger-mounted ultrasound probing device 100. In particular, FIGS. 4A and 4B depict the probe array 102 implemented within a glove 110 that makes up the finger-mounted ultrasound probing device 100. FIG. 4A depicts a palm view of the glove 110 and FIG. 4B depicts a back hand view of the glove 110. In accordance with an example embodiment of the present invention, the probe array 102 is integrated into the glove 110 and interconnected via integrated module wiring 112. Additionally, the module wiring 112 provides a connection between the probe array 102 and the ultrasonic system 200 via a connector 114. The connector 114 is configured as a conventional connector for use with traditional ultrasonic systems 200. As would be appreciated by one skilled in the art, the glove 110 can be constructed from any material known in the art capable of implementing the probe array 102. For example, the glove 110 can be a surgical grade glove to be utilized in a sterilized manner. Additionally, the glove 110 can be designed to be a reusable/serializable glove or a disposable glove.

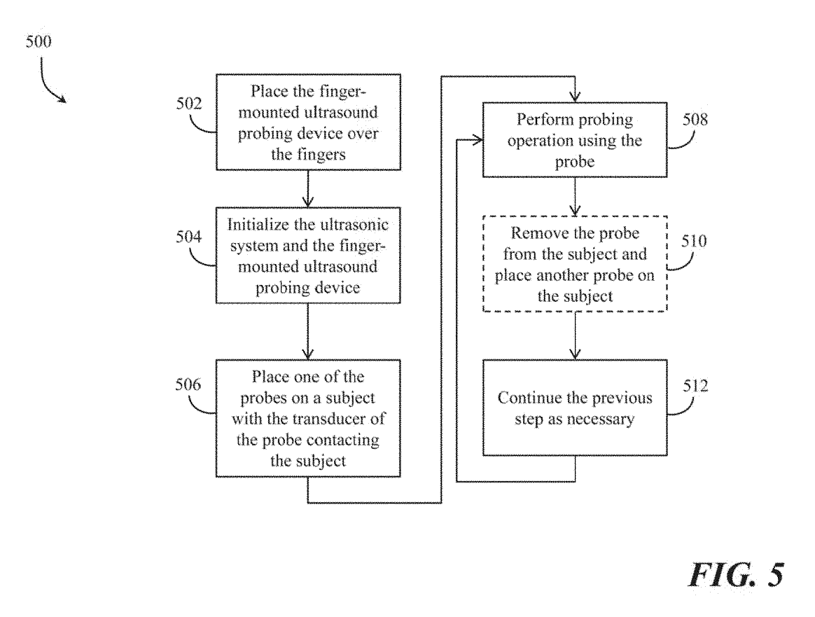

FIG. 5 depicts an exemplary flow chart showing the operation 500 of the finger-mounted ultrasound probing device 100, as discussed with respect to FIGS. 1A-4B. At step 502 the user places the finger-mounted ultrasound probing device 100 over their fingers. In particular, the user places each of the finger sheaths 106 of the probe array 102 over different fingers on the hand the user wants to use in performing the ultrasound probing. Depending on the implementation of the finger-mounted ultrasound probing device 100 (e.g., as depicted in FIGS. 3A and 3B or 4A and 4B) the user either places an entire glove 110 on the desired hand 108 or the probe array 102 on desired individual fingers. As would be appreciated by one skilled in the art, in the non-glove implementation, the probes 102a can be placed on any of the finger that is preferred by the user. For example, using a three finger sheath 106 implementation, the user can place the probes 102a on the index finger, the middle finger and the ring finger or the user can place the probes 102a on the middle finger, the ring finger, and the pinky finger. Similarly, the user can select finger sheaths 106 with the plurality of transducers 104 of different firing positions. For example, the user can combine finger sheaths 106 with side firing transducers of the plurality of transducers 104 (e.g., as depicted in FIG. 1A) with finger sheaths 106 with end firing transducers of the plurality of transducers 104 (e.g., as depicted in FIG. 1B)

At step 504 the user initializes the ultrasonic system 200 and the finger-mounted ultrasound probing device 100. The initialization can include powering on each respective device, securing connections between the devices (wired or wireless), and any other steps typically performed during an ultrasonic procedure. Additionally, in accordance with an example embodiment of the present invention, the initialization can include selecting the frequencies for each of the probes 102a within the probe array 102. For example, the user can modify the frequency and position (e.g., which finger) for each of the probes 102a within the probe array 102 by swapping out the plurality of transducers 104 and/or finger sheaths 106 within the probe array 102 with alternate transducers 104 and/or finger sheaths 106 of different frequencies.

At step 506 the user determines which one of the probes 102a is desirable to use at that point in time (e.g., based on frequency and/or finger position) and places the selected probe 102a into contact with the subject (e.g., a patient) to perform ultrasound imaging using that probe 102a. For example, if the user currently desires to obtain a shallow penetration and high resolution image, the user will select the probe 102a/finger containing a high frequency transducer of the plurality of transducers 104. At step 508 the user performs a desired probing operation using the selected probe 102a. As would be appreciated by one skilled in the art, this operation may include moving the finger with the desired probe 102a around in contact with the subject to obtain the desired image and image location.

Optionally, at step 510 the user can lift up the selected probe 102a (e.g., lifting the finger with the probe 102a located thereon) to break contact with the subject and select another probe 102a on another finger within the probe array 102 to place in contact with the subject. At step 512 the user continues the probing process of step 508 using the currently selected probe 102a and continue switching probes 102a as desired. In particular, during operation, a user continues switching which fingers are in contact with the subject and performing the ultrasound based on which frequency the user desires to utilize. As the user changes fingers, the corresponding frequency of the plurality of transducers 104 will be changed and the change in frequencies will be reflected on the ultrasound display device 204.

The functionality provided by the finger-mounted ultrasound probing device 100 enables a user to quickly change between transducer characteristics (e.g., between low resolution and high resolution imaging) without having to change a probe array or to another probe. Instead, the user merely switches between the probes 102a included within the probe array 102 by switching which finger is in contact with the patient. Accordingly, the user has access to multiple probe frequencies and switching between those frequencies all within a single hand device. This functionality enables the medical professional or other user to maintain proximity to a patient without having to adjust any ultrasonic equipment. The finger-mounted ultrasound probing device 100 can be utilized for improved ultrasound guided biopsy and surgery procedures by allowing medical personnel to conveniently switch the probes with different center frequency during the surgery without having to remove the probe hand from the patient. Eliminating the changing probes can greatly shorten the surgery time and decrease the possibility of cross contamination.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law. It is also to be understood that the following claims are to cover all generic and specific features of the invention described herein, and all statements of the scope of the invention which, as a matter of language, might be said to fall therebetween.

What is claimed is:

1. A finger-mounted ultrasound probe array, comprising:
a plurality of finger sheaths, each having a first end and a second end and each sized and dimensioned to fit over a finger of a user;
a plurality of transducers, each transducer coupled with each of the plurality of finger sheaths;
wherein each transducer of the plurality of transducers produces a different center frequency from the other of the plurality of transducers.

2. The probe array of claim 1, wherein the plurality of transducers are located proximate to a mid-region of each of the plurality of finger sheaths and are configured to fire and receive ultrasonic waves in a side firing orientation.

3. The probe array of claim 1, wherein the plurality of transducers are located proximate to the second end of each of the plurality of finger sheaths and are configured to fire and receive ultrasonic waves in an end firing orientation.

4. The probe array of claim 1, wherein the plurality of transducers comprise linear probes.

5. The probe array of claim 1, wherein the plurality of transducers comprise convex probes.

6. The probe array of claim 1, wherein the plurality of transducers comprise 2D matrix probes.

7. The probe array of claim 1, wherein the plurality of transducers comprise piezoelectric transducers or capacitive transducers.

8. The probe array of claim 1, wherein the plurality of finger sheaths are sized, dimensioned, and configured to slide over fingers of a user wearing a surgical glove.

9. The probe array of claim 1, wherein the plurality of finger sheaths are themselves portions of a surgical glove.

10. The probe array of claim 1, wherein each of the plurality of finger sheaths is distinct and separate from each other of the plurality of finger sheaths.

11. The probe array of claim 1, wherein each of the plurality of finger sheaths is fixed together.

12. The probe array of claim 1, further comprising communication hardware in communication with the probe array and configured for communication with an ultrasound machine using a wired or wireless form of communication.

13. The probe array of claim 1, wherein the probe array is adapted to implement ultrasound procedures during a biopsy procedure, or a tumor extracting procedure.

14. The probe array of claim 1, wherein each of the plurality of finger sheaths is removable and replaceable in real-time during use of the probe array to enable swapping in and out of transducers having different transducer characteristics.

15. The probe array of claim 1, wherein the plurality of transducers comprise high frequency transducers.

16. The probe array of claim 1, wherein the plurality of transducers comprise mid-range frequency transducers.

17. The probe array of claim 1, wherein the plurality of transducers comprise low frequency transducers.
